(19) United States
(12) Patent Application Publication
(10) Pub. No.: US 2008/0207994 A1
(43) Pub. Date: Aug. 28, 2008

(54) Endoscopic submucosal dissection using a high-pressure water jet

(75) Inventor: Bertrand Gonon, Ternay (FR)

Correspondence Address: LOUIS WOO;LAW OFFICE OF LOUIS WOO, 717 NORTH FAYETTE STREET, ALEXANDRIA, VA, 22314, US

(73) Assignee: Eschmann Holdsings Limited, West Sussex (GB)

(21) Appl. No.: 11/709,171

(22) Filed: Feb. 22, 2007

(51) Int. Cl. A61B 1/00

(52) U.S. Cl. 600/104

(57) ABSTRACT

The invention provides a method of performing a musectomy intervention which method comprises applying the following steps to a human or animal body having a mucosal region which needs to be removed:
injection of a physiologically acceptable liquid into the mucosal region from a high pressure source of said liquid;
dissection of the mucosal region; and
   removal of the dissected mucosal region; the method is advantageous because it has a reduced risk of perforation of the submucosal musculature.

The invention provides a method of performing an endoscopic submucosal dissection of the digestive tract using a high-pressure water jet.

Endoscopic mucosal resection (EMR) has become the standard endoscopic treatment for superficial neoplastic lesions of the digestive tract (strictly limited to the mucosa) such as:
dysplasias and superficial epidermal carcinomas of the oesophagus;
severe dysplasias or developed superficial adenocarcinomas on Barrett's oesophagus;
severe dysplasias or superficial adenocarcinomas of the stomach; and
superficial adenomas or adenocarcinomas of the duodenum, colon and rectum.

EMR has progressively replaced endoscopic techniques based on the destruction of the neoplastic tissue since, compared to these techniques, it enables a complementary histological analysis to be performed. This has two advantages in that it can be determined whether the endoscopic treatment is complete or partial, and the level of parietal invasion of the cancer can be analysed. These histological criteria are necessary for deciding whether the treatment should be exclusively endoscopic, or whether, on the contrary, a surgical treatment or a complementary radiotherapy treatment should be used.

The current EMR technique is generally known as mucosectomy. The simplest technique is to inject and cut and this forms the basis of all the other techniques.

This technique is preferably used for duodenal and colorectal lesions, and is always carried out using the following steps:
1. Identification of the lesion and precise determination of its size: this step is best carried out using vital stains, namely 2.5% Lugol for the epidermal oesophagus, and 0.5% indigo carmine for oesophageal adenocarcinomas and colon and gastric lesions. Staining is essential for flat lesions. After staining, the demarcation of the area to be resectioned is generally carried out by marking the contours of the lesion by means of electrocoagulation points. The depth of the area may be determined by high-frequency echoendoscopy;
2. Submucosal injection of physiological saline under the lesion, thereby forming a neopolyp (the first injection should enable the lesion to be better orientated by tilting it towards the endoscope): this step enables the musculature to be protected and assists the action of the cutting current;
3. Clamping of the lesion in the diathermy loop and checking its correct positioning by effecting pushing and pulling movements;
4. Cutting (by a pure high-frequency cutting current or endocutting) in the submucosal layer;
5. Removal of the piece using the loop or a threaded loop in the case of multiple fragments;
6. Removal, spreading and alignment of the piece or pieces on a flat support before fixing them for the histological analysis; and
7. Endoscopic verification that the resection is macroscopically complete since in the case of very large lesions it is often necessary to carry out resections in several fragments (piecemeal) or to destroy the residues by argon plasma coagulation.

However, these mucosectomy techniques are rarely satisfactory. Ideally, it would be desirable if the resection of even large lesions could be carried out in a single piece with a lateral safety margin of 2 mm and at a depth in the submucosal layer. This is because it has been shown that the risk of recurrence of lesions after mucosectomy is greater in the case where resection is carried out in several pieces and the safety margin is not observed. The level of recurrence is zero if this safety margin is observed, is about 20% if it is not, and is 50% if the cutting edge is reached. However, the use of a diathermy loop, even after submucosal injection, does not provide such a result: the resections are often performed piecemeal and not en bloc, and very often without any lateral safety margin. Moreover, the safety margins, where they exist, are often reduced or are indeterminable on account of artefacts associated with the electro-coagulation. For these reasons the Japanese authors have developed techniques for step-by-step submucosal dissection (SMD) and resection en bloc.

From the use of SMD, there is a better carcinological monitoring and a histological reading of improved quality for monitoring the complete or partial appearance of the resection.

These techniques adapted to large size lesions use diathermy instruments (electrosurgical knives) derived from celioscopy: needle knife, hook knife, flex knife, triangle-tip knife, insulated-tip knife (or IT knife which is a pointed surgical knife provided at its end with a small ceramic insulating ball that protects the musculature from diffusion of the current).

The SMD technique is based on mucosectomy and involves the following steps:
1. Submucosal injection of physiological saline or a higher viscosity injection product (for example sodium hyaluronate, dihydroethylcellulose and polyethylene glycol). The advantage of using a higher viscosity injection product is that the separation may be more permanent;
2. Circumferential mucosal incision around the lesion: first using a needle knife to penetrate the mucosal wall, followed by introduction of an IT knife into the orifice so as to effect the circumferential cutting by an endocutting current (3, 120 W);
3. Submucosal dissection of the piece: either by means of a loop inserted into the cutting groove, for less extensive lesions; or progressively with an IT knife by making tangential incisions with respect to the submucosa (it is absolutely necessary to have a good separation for this method);
4. Removal of the piece in one part.

The major SMD resection is particularly suitable for the ablation of gastric neoplastic lesions and also of colon lesions. Furthermore, they can be adapted to extensive and circumferential oesophageal mucosal resection, as has been demonstrated in two experimental studies carried out on pigs (Hino S et al: “A new material to aid endoscopic mucosal resection”; Gastrointest Endosc 2003; 57:AB1 and Rajan E et al: “Widespread endoscopic mucosal resection of the esophagus in a porcine model using a prototype endoscopic cap device”; Gastrointest Endosc 2003; 57: AB1). This is regarded as a more attractive procedure than circular oesophageal mucosectomy using a transparent cap, since it permits en bloc resections up to and including the sub-cardial region.

This technique effectively permits a surgical dissection involving removal of a single piece of tissue with safety margins, but has three disadvantages: it is very long (on average 90 minutes for pieces 2 to 3 cm in diameter), the technique is difficult to learn since only the Japanese have mastered it with satisfactory results (Rösch T et al: “Attempted endoscopic en bloc resection of mucosal and submucosal tumours using insulated-tip knives: a pilot series”; Endoscopy 2004; 36(9); 788-801), and it is accompanied by a high level of complications (1 to 8% of secondary haemorrhages and 4 to 8% of perforations depending on the dissection site (Oda I et al: “Endoscopic submucosal dissection for early gastric cancer: technical feasibility, operation time and complications from a large consecutive series”; Dig. Endosc. 2005; 17; 54-58)). In addition, it does not avoid the difficulties of a histological analysis of the resection margins on account of artefacts associated with the electrocoagulation.

A way of ameliorating these problems has been sought.

According to the invention there is provided a first method of performing a musectomy intervention which method comprises applying the following steps to a human or animal body having a mucosal region which needs to be removed:
an injection step comprising injection of a physiologically acceptable liquid into the mucosal region from a high pressure source of said liquid;
an incision step comprising mucosal incision around the mucosal region;
a dissection step comprising dissection of the mucosal region; and
a removal step comprising removal of the dissected mucosal region.

According to the invention there is further provided a second method of performing a musectomy intervention which method comprises applying the following steps to a human or animal body having a gastric mucosal region which needs to be removed:
an injection step comprising injection of a physiologically acceptable liquid into the gastric mucosal region from a high pressure source of said liquid;
an incision step comprising mucosal incision around the gastric mucosal region;
a dissection step comprising dissection of the gastric mucosal region using electrosurgery; and
a removal step comprising removal of the dissected gastric mucosal region.

According to the invention, there is also provided a third method of performing a musectomy intervention which method comprises applying the following steps to a human or animal body having an oesophageal mucosal region which needs to be removed:
an injection step comprising injection of a physiologically acceptable liquid into the oesophageal mucosal region from a high pressure source of said liquid;
an incision step comprising mucosal incision around the oesophageal mucosal region;
a dissection step comprising dissection of the oesophageal mucosal region using a high-pressure jet of a physiologically acceptable liquid; and
a removal step comprising removal of the dissected oesophageal mucosal region.

It has surprisingly been found possible to apply the submucosal dissection (SMD) technique using a high pressure water jet. This technique is widely used in various surgical fields: hepatic surgery (hepatectomy, liver transplants, etc.), urological surgery (partial nephrectomy, etc.), gynaecological surgery, surgery of the digestive tract, and general surgery; it is currently under experimental and clinical evaluation in other surgical fields and medical interventions, including neurosurgery and cardiological and radiological procedures.

The advantages of the invention include:
the simplicity and speed of the procedure;
the reduction of risks of perforation as regards the musculature, which is more resistant than the submucosal layer to the water jet;
reduction of risk of burns and necrosis;
the suppression of coagulation lesions;
potentially improved control of haemostasis with respect to the blood vessels, which may be preventively coagulated with heated forceps; and
a better histological analysis of the cutting plane, on account of the absence of artefacts associated with electrocoagulation.

The method of the invention may be applied to a gastric, rectal, thoracic, urological, gynaecological or oesophageal mucosal region.

In the method of the invention, the injection step may comprise submucosal injection. The incision step may comprise circumferential incision. The dissection step may comprise submucosal dissection of the mucosal region.

In some aspects, the dissection step may comprise use of a high-pressure jet of a physiologically acceptable liquid. In some aspects, the high pressure source of physiologically acceptable liquid used in the invention may be a generator-distributor as described in U.S. Pat. No. 6,083,189, U.S. Pat. No. 6,423,028, and/or U.S. Pat. No. 6,322,533, the contents of each of which documents are incorporated herein by reference.

In some aspects, the dissection step may comprise use of electrosurgery. The electrosurgery may comprise use of electrosurgical cutting or electrosurgical coagulation.

Where the method according to the invention comprises the use of electrosurgery, a combined electrosurgery and waterjet handpiece may be used. A suitable combined handpiece is described in co-pending U.S. patent application having U.S. Ser. No. 11/640,328 filed on 18 Dec. 2006, the contents of which are incorporated herein by reference.

The injection step may comprise use of a catheter with an injecting end tool such as that described in co-pending U.S. patent application having U.S. Ser. No. 11/055,087 filed on 11 Feb. 2005, the contents of which are incorporated herein by reference.

The dissection step may comprise performing a haemostasis coagulation to prevent bleeding. Optionally, the haemostasis coagulation is performed using heated forceps.

The physiologically acceptable liquid used in the invention may be saline. Alternatively, the physiologically acceptable liquid may be a high viscosity injection product, for example, sodium hyaluronate, dihydroethylcellulose, polyethylene glycol or the like. The physiologically acceptable liquid may optionally comprise a pharmaceutically active substance. In some aspects, the volume of physiologically acceptable liquid injected in the injection step is controlled.

In some aspects, the method of the invention may comprise the further step of incision around the mucosal region before the dissection step.

The method of the invention may be applied to a human or animal body. In some aspects, the animal may be a mammal.

The invention will now be illustrated with reference to the following Example which is not intended to limit the scope of the invention claimed.

### EXAMPLE

In the following Example, methods according to the invention were carried out in comparison with the SMD reference method, of Japanese origin using twelve anaesthetised pigs. The object of this experimental study was to investigate all the potential benefits (speed, absence of side effects, better histological analysis) of SMD assisted by a WaterJet® (Eschmann Equipment, Lancing, West Sussex BN15 8TJ, UK) on a porcine model. The WaterJet® is a water compressor which can reach pressure of up to 30 bars and is used with a flexible PVC catheter which can transmit the water jet under high pressure without deformation or loss of pressure.

12 Pigs weighing 25 to 35 kg, anaesthetised according to the ESI protocol and ventilated were involved in the experimental study.

The equipment used was as follows:
Simple operator channel endoscope with video-endoscopy column (Olympus);
A WaterJet® (Eschmann Equipment Limited, UK) as a continuous jet for the submucosal injection with an adjustable preset volume (0 to 100 ml), and a continuous or pulsed jet for the submucosal dissection;
A flexible catheters with a retractable needle to guarantee high pressure;
A set of electrocoagulation instruments as used in endoscopy for SMD (flex knife, IT knife, hook knife, triangle-tip knife, loop) as well as heated forceps (instruments necessary for the thermal endoscopic haemostasis);
An electrically operated surgical knife;
Injection instruments: for the haemostasis by adrenaline and for the submucosal injection; and
A reusable clip-type forceps, with 50 loadable clips.

Three different methods of submucosal dissection were compared; the Japanese reference method which uses electrocoagulation, and two new methods which both use the WaterJet® for the submucosal injection with one also using the WaterJet® for the submucosal dissection.

Each pig underwent three gastric resections, namely one resection per method, followed by an oesophageal resection, varying the method used each time.

Each pig fasted overnight except for water, according to the ESI protocol; was anaesthetised with ketamine, propofol and sufentanyl and then each pig was intubated or tracheotomised and ventilated.

Dissection of three pieces of stomach followed by one piece of oesophagus, using the three different methods employed in succession (the different times of each resection were measured and a video recording was made). The steps used were as follows:
a. Demarcation of the mucosal region to be resectioned, by a peripheral marking of several coagulation points made with the tip of a loop.
b. Submucosal injection of physiological saline using an injection needle so as to lift the mucosa of the deep-lying layer:
   either manually using a syringe in method 1 (reference method); or
   by the WaterJet® (adjusted to 10 bars) in methods 2 and 3.
c. Circular incision around the mucosal region to be removed (thus around the marking points), by electrocoagulation with an endocutting current (3, 120 W) using one of the various electrocoagulation instruments (this is the only step common to all three methods).
d. Submucosal dissection between the mucosa and the musculature:
   either in the case of the methods 1 and 2 by electrocoagulation by means of the electrocoagulation instruments, using an endocutting current (3, 120 W); or
   in the case of method 3 by means of the WaterJet® catheter, with the WaterJet® adjusted to 20 bars, in the form of a discontinuous (pulsed) jet.
   The vessels isolated in the course of the dissection were coagulated progressively by simple coagulation with the electrocoagulation instruments or using heated forceps, with a low coagulation current (40 to 50 W), this procedure being adopted in all three cases.
e. Removal of the dissected pieces of mucosa, followed by spreading the pieces out on a plate before fixing them in formol. Precise measurements of their sizes enabled us to calculate their circumference and surface area, and thus the different submucosal injection rates, submucosal dissection and overall resection expressed in cm² per minute, as well as the circumferential incision rate expressed in cm per minute.

The following criteria were used to evaluate the dissection by the operators:
a) Speed of the dissection: by calculating the dissection speeds.
b) Ease of the dissection by the operator: by an analog visual scale (AVS) for the circumferential incision and submucosal dissection phases.

The analysis of the results of the experimental study has been divided into two parts. The first part comprises the results from the resection of 36 pieces of gastric mucosa. The data obtained is listed in Table 2 (statistical evaluation by the Student t-test):

TABLE 2

| | METHOD 1 (ref) | | METHOD 2 | | METHOD 3 |
|---|---|---|---|---|---|
| GASTRIC RESULTS | Manual injection | | injection by WaterJet | | injection by WaterJet |
| Mean Standard | followed by dissection by | | followed by dissection by | | followed by dissection by |
| deviation (max–min) | electrocoagulation | | electrocoagulation | | WaterJet |
| Rate of submucosal | 5.58 | P < 0.05 | 10.19 | | 9.29 |
| injection in cm²/min | 2 (9.17–2.52) | | 5.88 (26.29–4.82) | | 8.51 (31.76–2.33) |
| Rate of circumferential | 1.4 | | 2.38 | | 2.33 |
| incision in cm/min | 0.68 (2.62–0.61) | | 1.89 (7.28–0.29) | | 1.83 (6.35–0.41) |
| Rate of submucosal dissection | 1.79 | | 2.18 | P < 0.05 | 0.75 |
| in cm²/min | 0.91 (3.31–0.3) | | 1.35 (4.4–0.38) | | 0.38 (1.44–0.32) |
| AVS of the circumferential | 5.9 | | 7.1 | | 7.2 |
| incision | 2 (8.5–3) | | 3.1 (10–1) | | 2.3 (10–2) |
| AVS of the submucosal | 7.3 | | 8.3 | P < 0.05 | 6.8 |
| dissection | 1.9 (10–4) | | 1.6 (10–6) | | 1.4 (8–4) |
| Number of necessary | 2/12 | | 0/12 | | 0/12 |
| reinjections | | | | | |
| Number of uses of | 7/12 | | 1/12 | | 2/12 |
| an IT knife for the circumferen | | | | | |
| incision | | | | | |
| Number of uses of the loop to | 1/12 | | 1/12 | | 1/12 |
| complete the reception | | | | | |
| Complete en bloc resections | 11/12 | | 11/12 | | 12/12 |
| Number of damaged pieces | 0/12 | | 1/12 | | 3/12 |
| Perforations | 0/12 | | 0/12 | | 1/12 |
| | | | (manipulation error) | | |
| Macroscopic appearance of | 1 compacted haematoma | | 1 diffuse haematoma | | 1 5 mm-size perforation |
| the musculature | | | | | |
| Success | 11 | | 11 | | 11 |

### Analysis of the Data in Table 2

An examination of these results shows that the quickest and easiest method to use is the second method since it combines two advantages: a quick and very efficient submucosal injection (without the need for reinjection), allowing a circular incision without any difficulty of the mucosa, without having to use an IT knife (this instrument slows down the cutting speed due to its insulated tip). Secondly, a submucosal dissection by electrocoagulation with an IT knife, which in the context of this application is a very precise and quick instrument provided that there is a good separation.

As regards the complications, there was a single gastric perforation during the first procedure carried out by operator 1 due to an incorrect movement of the catheter during the third method (which is therefore not directly responsible).

Finally, there was only one failure with each method: for the methods 1 and 2 this involved an incomplete resection (the whole set of marking points was not found), and for method 3 this involved a perforation due to a manipulation error (which was successfully corrected using clips).

The second part of the results of the experimental study comprises results from the resection of 12 circumferential pieces of oesophageal mucosa which was carried out by a single operator. The data are shown in Table 2 (statistical evaluation by the Student T-test):

TABLE 3

| | METHOD 1 (ref) | | METHOD 2 | METHOD 3 |
|---|---|---|---|---|
| OESOPHAGEAL RESULTS | Manual injection followed | | injection by WaterJet | injection by WaterJet |
| Mean | by dissection by | | followed by dissection by | followed by dissection by |
| Standard deviation (max–min) | electrocoagulation | | electrocoagulation | WaterJet |
| Rate of submucosal injection | 2.52 | | 5.76 | 7.55 |
| in cm²/min | 1.57 (3.89–0.39) | | 2.09 (8.36–4.06) | 3.86 (12.11–2.7) |
| Rate of circumferential | 0.67 | | 0.99 | 1.44 |
| incision in cm/min | 0.11 (0.79–0.54) | | 0.35 (1.36–0.66) | 1.01 (2.89–0.71) |
| Rate of submucosal | 0.69 | | 1.85 | 2.41 |
| dissection in cm²/min | 0.38 (1.11–0.28) | | 0.99 (2.71–0.89) | 1.9 (4.08–0.69) |
| AVS of the lower circumferential | 2.8 | P < 0.05 | 7.6 | 6.5 |
| incision | 2.2 (5–0) | | 2.1 (9.5–5) | 3.9 (10–1.5) |
| AVS of the upper circumferential | 4.8 | P < 0.01 | 8.9 | 9.1 |
| incision | 1.7 (7–3) | | 1.3 (10–7) | 0.9 (10–8) |
| AVS of the submucosal | 4.5 | P < 0.05 | 8.9 | 8 |
| dissection | 1.3 (6–3) | | 1.3 (10–7) | 2.4 (10–5) |
| Perforations | 1/4 | | 0/4 | 1/4 |
| | (injection fault) | | | (error in the lower circumfere |
| | | | | incision with the hook knife) |
| Macroscopic appearance of | 1 10 mm-size perforation | | | 1 15 mm-size perforation |
| the musculature | | | | |
| Success | 2 | | 4 | 3 |
| | (failure: 1 perforation | | | (failure: 1 perforation) |
| | and 1 undiscovered | | | |
| | perforation) | | | |

### Analysis of the Data in Table 3

These results show that the methods 2 and 3 are the easiest to use and are also the quickest; method 3 being the most advantageous. This is explained by the quality of the submucosal injection in these two methods, enabling the oesophageal mucosa to be dissected by progressively separating it. In addition, in method 3, the sites where the separation is not sufficient are rapidly dissected by the high-pressure water jet, which is ideally aimed so as to strike the wall tangentially, which satisfactorily completes the injection.

The learning curves of the different methods in terms of speed and ease of use are very clearly in favour of the third method as opposed to the first method, which is slow; the second method also exhibits good features, though to a lesser extent than the third method.

As regards complications, there were two cases of perforations, in each case in the first series of procedures for the methods 1 and 3, and specifically during the lower circumferential incision phase. The basic problem of the first method was a faulty injection (the musculature was not sufficiently spaced from the mucosa during the use of the Hook knife to make the first incision, which is directly attributed to the technique). In the context of method 3, the separation was perfect, but unfortunately a manipulation error, which is not directly attributable to the technique, occurred in the same phase with the hook knife.

The macroscopic appearance of the musculature allowed the two already described perforations to be revealed, which exhibited a 10 mm-size opening in the first method and a 15 mm-size opening in the third method.

### CONCLUSION

The most effective and the least invasive method for resection of the gastric mucosa is that using injection by the WaterJet® and dissection by electrocoagulation (method 2). It was found that a high flow rate injection creates a cleavage plane by dilacerating the submucosa, thereby facilitating the circumferential incision and the submucosal dissection by electrocoagulation, without any need for reinjection.

As regards circumferential resection of the oesophageal mucosa, the method that provides the best results is that using the WaterJet® for the injection and for the dissection (method 3); the high flow rate injection effectively produces a cleavage plane by dilacerating the submucosa, which facilitates the upper and lower circumferential incisions, but above all the high pressure water jet catheter permits a gentle oesophageal submucosal dissection by parting the mucosa step-by-step while permitting the preventive coagulation of the vessels, which are thereby easier to see. The riskiest and most intricate phase of this resection is the lower circumferential incision, which could be replaced by a sub-cardial circumferential incision using a mirror, so as to be in the correct position underneath the mucosal junction and thereby avoid contractions of the cardia during the use of the cutting currents.

The two applications of the WaterJet® have clearly demonstrated their value in SMD, for the high flow rate submucosal injection and for the high pressure water jet dissection in the case of the oesophagus. It is believed that, although the high pressure water jet dissection did not proved superior in the case of gastric resections, it could be useful for facilitating electrocoagulation in the case of a poor exposure of the submucosa, so as to combine a dissection and reinjection procedure in a single stage.

What is claimed is:

1. A method of performing a musectomy intervention which method comprises applying the following steps to a human or animal body having a mucosal region which needs to be removed:
an injection step comprising injection of a physiologically acceptable liquid into the mucosal region from a high pressure source of said liquid;
an incision step comprising mucosal incision around the mucosal region;
a dissection step comprising dissection of the mucosal region; and
a removal step comprising removal of the dissected mucosal region.

2. A method as defined in claim 1 wherein the mucosal region is a gastric, rectal, thoracic, urological, gynaecological or oesophageal mucosal region.

3. A method as defined in claim 1 wherein the injection step comprises submucosal injection.

4. A method as defined in claim 1 wherein the incision step comprises circumferential incision.

5. A method as defined in claim 1 wherein the dissection step comprises submucosal dissection of the mucosal region.

6. A method as defined in claim 1 wherein the dissection step comprises use of a high-pressure jet of a physiologically acceptable liquid.

7. A method as defined in claim 1 wherein the dissection step comprises use of electrosurgery.

8. A method as defined in claim 7 which comprises the use of a combined electrosurgery and waterjet handpiece in the injection and dissection steps.

9. A method as defined in claim 1 wherein the dissection step comprises performing a haemostasis coagulation to prevent bleeding.

10. A method as claimed in claim 1 wherein the physiologically acceptable liquid is saline.

11. A method as claimed in claim 1 wherein the physiologically acceptable liquid is a high viscosity injection product.

12. A method as claimed in claim 1 which comprises the further step of incision around the mucosal region before the dissection step.

13. A method of performing a musectomy intervention which method comprises applying the following steps to a human or animal body having a gastric mucosal region which needs to be removed:
an injection step comprising injection of a physiologically acceptable liquid into the gastric mucosal region from a high pressure source of said liquid;
an incision step comprising mucosal incision around the gastric mucosal region;
a dissection step comprising dissection of the gastric mucosal region using electrosurgery; and
a removal step comprising removal of the dissected gastric mucosal region.

14. A method of performing a musectomy intervention which method comprises applying the following steps to a human or animal body having an oesophageal mucosal region which needs to be removed:
an injection step comprising injection of a physiologically acceptable liquid into the oesophageal mucosal region from a high pressure source of said liquid;
an incision step comprising mucosal incision around the oesophageal mucosal region;
a dissection step comprising dissection of the oesophageal mucosal region using a high-pressure jet of a physiologically acceptable liquid; and
a removal step comprising removal of the dissected oesophageal mucosal region.
